(19) United States
(12) Patent Application Publication
(10) Pub. No.: US 2002/0182685 A1
(43) Pub. Date: Dec. 5, 2002

(54) In vivo generation of antibodies against antigens secreted in a host by transfected MEL cells

(75) Inventors: Maurice Ronald Charles Needham, Congleton (GB); Melvin Hollis, Congleton (GB)

Correspondence Address: JANIS K. FRASER, PH.D., J.D., Fish & Richardson P.C., 225 Franklin Street, Boston, MA, 02110-2804, US

(73) Assignee: Zeneca Limited, a British corporation

(21) Appl. No.: 10/193,378

(22) Filed: Jul. 11, 2002

Related U.S. Application Data
(63) Continuation of application No. 08/913,790, filed on Sep. 19, 1997, now abandoned.
Application No. 08/913,790 is a 371 of international application No. PCT/GB96/00616, filed on Mar. 18, 1996.

(30) Foreign Application Priority Data
Mar. 22, 1995 (GB) 9505777.4

(51) Int. Cl. C12P 21/04; A01K 67/027

(52) U.S. Cl. 435/70.21; 800/6

(57) ABSTRACT

A method for raising antibodies in a mammalian host using live cells transfected with a biological of interest.

### CROSS-REFERENCE TO RELATED APPLICATIONS

This application is a continuation of, and claims benefit of, U.S. application Ser. No. 08/913,790, filed Sep. 19, 1997, which claims benefit of International Application PCT/GB96/00616, filed Mar. 18, 1996, which claims benefit of Great Britian application Serial No. 9505777.4, filed Mar. 22, 1995, all of which are incorporated by reference in their entirety.

The present invention relates to methods for the generation of antibodies. In particular it relates to the use of live, secretory cells in such methods.

Available methods for the production of antibodies to a biological of interest are involved and have certain disadvantages. In particular they require the production of sufficient purified material to carry out the immunisation of animals. This may involve the use of cell cultures or tissues, fermentation broths, protein isolation and extraction from gels, affinity chromatography and other procedures.

We have now found that antibodies can be successfully raised in mice which have been injected with transfected murine erythroleukaemia (MEL) cells. This was gratifying since it was unclear whether or not the cells expressing the recombinant protein, or the expressed protein itself would remain in the tissue for sufficient time for a significant immune response to occur. Failure to observe tumour formation in animals which had been injected with the recombinant cells indicated that the cells at least were being rapidly cleared from the animal tissues.

Therefore in a first aspect of the invention we provide a method for the generation in a mammalian host of antibodies to a biological of interest which method comprises incorporating murine erythroleukaemia (MEL) cells comprising DNA encoding secrete the biological of interest, allowing the host to raise antibodies thereto, and isolating said antibiodies from the host.

The mammalian host is conveniently a non-human animal such as a rat or a mouse, preferably a mouse.

The biological of interest comprises any convenient protein to which antibodies may be raised. In particular it may be a protein to which is difficult to raise or purify antibodies using presently available technology. Such difficulties may also arise by virtue of the tissue or species of origin, for example proteins of human origin. It is conveniently introduced into the murine erythroleukaemia (MEL) cells via transfection with a vector.

Convenient vectors include the pEV expression vector. The pEV expression vector is a modified version of the previously reported MEL expression vector pGSE1417/EC3 (Needham et al., Nucleic Acids Research, 1992, 20 (5), 997-1003). pEV was generated as follows:

The EcoRI site in the thymidine kinase promoter, the Bg1II site in the neomycin resistance gene and the NotI site in the LCR of pGSE1417 (20) were sequentially deleted by restriction enzyme digestion followed by T4 polymerase treatment and religation to generate pDGSE1417. The ClaI-Asp718 fragment from pEC3 (1) was ligated between the ClaI and Asp718 sites of pDGSE1417 to generate pEV.

DNA encoding the biological of interest is introduced into the vector using conventional molecular biology techniques for example as disclosed by Maniatis et al in Molecular Cloning, A Laboratory Manual.

The transfected MEL cells are conveniently incorporated into the mammalian host by injection, for example in the form of a bolus to be introduced subcutaneously or into the peritoneal cavity.

The polyclonal antibodies are conveniently harvested from serum obtained from the mammal. The use of splenic lymphocytes from animals immunised in this way can, via cell fusion, give rise to monoclonal antibody producing cell lines. Such polyclonal and monoclonal antibodies represent further and independent aspects of the invention.

The invention will now be illustrated but not limited by reference to the following Examples:

### EXAMPLE 1

Generation of Anti-VCAM Antibodies Using Recombinant MEL Cell Immunisation.

Modification of VCAM-1 cDNA:

VCAM-1 cDNA (Lobb et al., Biochem. and Biophys. Res. Comms., 1991, 178 (3), p1498-1504) was obtained from British Biotechnology and was modified by polymerase chain reaction (PCR) procedures to:

1) Remove unwanted 5′ non-coding sequence.

2) Include a consensus translation initiation (C.T.I.) sequence immediately upstream of the translational start site (5′ methionine).

(N.B. In addition this sequence was omitted for the purpose of baculovirus expression).

3) Terminate translation at the amino acid prior to the start of the transmembrane region (by introducing a translational stop codon at amino acid 72).

4) Include restriction enzyme sites at each end of the VCAM-1 cDNA to enable sub-cloning into a range of expression vectors.

MEL Cell Expression of sVCAM-1:

The modified sVCAM-1 cDNA was transferred into the MEL cell expression vector pEV (Needham et al., 1995, Protein Expression and Purification, 6 (2), 1995). MEL C88 cells (Deisseroth et al, Cell, 1978, 15, 55-63) were transfected with the pEV/sVCAM-1 expression construct. Six single cell clones and a population representing >200 clones were isolated, induced and tested for sVCAM-1 expression by VCAM-1 enzyme linked immunosorbent assay (ELISA). Initial ELISA results indicated that sVCAM-1 protein was expressed at between 1 and 5 mg/ml. One clone (*12) which expressed sVCAM-1 at 5 mg/ml was selected for further studies.

Antibody Generation:

MEL/sVCAM-1 clone * 12 cells expressing were washed free from any residual culture medium using phosphate buffered saline (PBS) and then centrifuged to a soft pellet using a bench-top centifuge. The resultant slurry was diluted with fresh PBS to give a final concentration of 5×107 cells per 100 uls. 100 uls of cell suspension was injected as a subcutaneous bolus into the posterior flank of each of a group of 12 week old female Balb/C mice. This dose was repeated after 21 days and again after a further 14 days. Blood samples were taken on days 35 and 65.

Solid phase enzyme linked immunosorbent assay (ELISA) of pooled serum samples using purified recombinant VCAM protein as antigen gave indicated 50% binding titres of:

1:1400 at 35 days and

1:6000+ at 65 days

indicating that anti-VCAM polyclonal antibodies had been generated in the test animals.

12 weeks after the 3rd dose, the mice were each given a booster injection of 20 ugs of purified recombinant VCAM protein contained in 100 uls of PBS. 4 days later the mice were humanely culled and splenectomised and the splenocytes from 4 of their spleens used in fusion experiments with the NS0 myeloma cell line (Methods in Enzymology, 1981, 73B:3). 2316 culture wells were set up following the fusion process and after biochemical selection using HAT medium (Littlefield, Experimental Cell Research, 1966, 411, 190) and growth in culture, 95 of these produced growing monoclonal cell colonies. Supernatant culture medium which was positive for anti-VCAM activity as judged by ELISA was produced by 16 of the 95. 12 of the 16 were recloned, by limiting dilution and 5 of the 12 produced clones which were postive for anti-VCAM activity.

Examples of the positive clones were grown up to produce frozen stocks of the cell lines and exhausted supernatant from these cell lines was tested by ELISA and Western Blot analysis to confirm the continuance of monoclonal antibody activity.

### EXAMPLE 2

Generation of Monoclonal Anti-Monocyte Chemoattractant Protein-1 (MCP 1) Antibodies by Recombinant MEL Cell Immunisation

Modification of MCP-1 cDNA:

MCP-1 cDNA was isolated and modifed for expression as reported by Needham et al., Protein Expression and Purification, 1996, 7, 173-182.

Mel Cell Expression MCP-1 cDNA

The modified MCP-1 cDNA was transferred into the MEL cell expression vector pEV (Needham et al., 1995, Protein Expression and Purification, 6 (2), 1995). MEL C88 cells (Deisseroth et al, Cell, 1978, 15, 55-63) were transfected with the pEV/MCP-1 expression construct. Six single cell clones and a population representing >200 clones were isolated, induced and tested for MCP-1 expression by Northern blot, calcium flux and chemotaxis assays and by mass spectrometry (Needham et al., Protein Expression and Purification, 1996, 7, 173-182). Results indicated that MCP-1 protein was expressed at between 1 and 5 mg/ml. One clone (*5) which expressed MCP-1 at >5 mg/ml was selected for further studies.

Antibody Generation:

This was performed exactly as for VCAM in Example 1 except that for antibody generation, the rest period between the last MEL cell injection and the pre-fusion booster was reduced to six weeks. Also 480 culture wells were set up after the fusion process and 35 of these produced growing monoclonal cell colonies. These eventually gave rise to 3 monoclonal antibody producing lines.

1. A method for the generation in a mammalian host of antibodies to a recombinant protein of interest which method comprises inducing murine erythroleukaemia (MEL) cells comprising DNA encoding the recombinant protein of interest to express the recombinant protein of interest via exposure to an inducing agent in a culture medium, selecting MEL cells that express the recombinant protein of interest, washing the selected MEL cells to remove said culture medium, introducing the selected MEL cells into the mammalian host, allowing the MEL cells to express and secrete the recombinant protein in the mammalian host, allowing the mammalian host to raise antibodies thereto, and isolating said antibodies from the host.

2. A method as claimed in claim 1 wherein the mammalian host is a non-human animal.

3. A method as claimed in claim 1 wherein DNA encoding the biological of interest is introduced into the MEL cells via transfection with a vector.

4. A method as claimed in claim 1 wherein the MEL cells are injected into the mammalian host.

5. A method as claimed in claim 4 wherein the MEL cells are injected in the form of a bolus to be introduced subcutaneously or into the peritoneal cavity.

6. A method as claimed in claim 2, wherein the mammalian host is a rat or a mouse.
